(19) United States
(12) Patent Application Publication
(10) Pub. No.: US 2024/0342436 A1
(43) Pub. Date: Oct. 17, 2024

(54) INTERLOCK ASSEMBLY FOR A MICROCATHETER DEVICE

(71) Applicant: SCIENTIA VASCULAR, INC., West Valley City, UT (US)

(72) Inventors: Nolan LESUEUR, West Valley City, UT (US); Clark DAVIS, Holladay, UT (US)

(21) Appl. No.: 18/633,424

(22) Filed: Apr. 11, 2024

Related U.S. Application Data
(60) Provisional application No. 63/459,119, filed on Apr. 13, 2023.

(51) Int. Cl. A61M 25/01; A61M 25/00

(52) U.S. Cl. CPC A61M 25/0108; A61M 25/0138; A61M 2025/0042

(57) ABSTRACT

Disclosed is an interlock assembly for a catheter with features that provide an improved connection between a marker band and microfabricated tube. The microfabricated tube features radial apertures that reduce the outer diameter of the distal region of the catheter, allow the catheter to fit into smaller size vessels, and permit an outer laminate to flow into the apertures while still having a large structural shape to interlock with the marker band.

### CROSS-REFERENCE TO RELATED APPLICATIONS

This application claims priority to and the benefit of U.S. Provisional Patent Application No. 63/459,119, filed Apr. 13, 2023 and titled “Interlock Assembly for a Microcatheter Device”, the entirety of which is incorporated herein by reference.

### BACKGROUND

Interventional devices such as guidewires and catheters are frequently utilized in the medical field to perform delicate procedures deep within the human body. Typically, a catheter is inserted into a patient's femoral, radial, carotid, or jugular vessel and navigated through the patient's vasculature to the heart, brain, or other targeted anatomy over a guidewire. Once in place, the catheter can be used to deliver drugs, stents, embolic devices, radiopaque dyes, or other devices or substances for treating the patient in a desired manner.

In many applications, such an interventional device must be navigated through the tortuous bends and curves of a vasculature passageway to arrive at the targeted anatomy. Such an interventional device requires appropriate placement within the human body to treat the patient. A marker band is a catheter component used in such applications to aid with guidance and placement of catheter devices during medical procedures. Marker bands can include radiopaque material so that they appear under fluoroscopy to inform medical professionals of the catheter orientation and placement.

Existing marker bands are usually held in place by crimping a C-shaped radiopaque ribbon around a polytetrafluoroethylene (PTFE) liner and then laminating a polymer over the perimeter of the ribbon to fully enclose the component. However, these marker bands have been known to dislodge themselves from the enveloping polymers and migrate into the patient's vasculature. Marker bands that dislodge into the patient's vasculature can lead to seriously bodily injury and death.

Additionally, most other catheters on the market have problems with the distal tip of the catheter ovalizing or becoming flat. This happens due to the low hoop strength of the commonly used braid or coil structures of current catheters, which often include a soft platinum-iridium crimped marker band ring or marker band ring of similar soft, radiopaque material.

Difficulties thus exist in guiding the catheter and aligning its distal tip with the targeted anatomy. Accordingly, there is a need for an improved connection to secure marker bands to the catheter and prevent dislodgement. There is also a need for an improved distal end of a catheter that prevents ovalization and undesirable deformation. A catheter with such components would be safer, more predictable, and more versatile than current marker bands for guidewires and catheters.

### SUMMARY

The present disclosure relates to an interlock assembly for a catheter. The interlock assembly includes: a microfabricated tube including a distal ring, the distal ring including at least one ring tenon and at least one ring mortise; and a marker band including at least one band tenon and at least one band mortise. The at least one ring mortise is configured to receive and interlock with the at least one band tenon and the at least one band mortise is configured to receive and interlock with the at least one ring tenon. The microfabricated tube can include a fenestrated segment extending from a main segment of the microfabricated tube to the distal ring of the microfabricated tube.

In some embodiments, the fenestrated segment is a helical segment. The distal ring can be integrally connected to the helical segment to form a monolithic structure.

The marker band can include at least two band mortises and the distal ring can include at least two ring tenons, and the marker band can include at least two band tenons and the distal ring can include at least two ring mortises.

At least one ring tenon and/or at least one band tenon can include an aperture to allow material of an outer laminate to extend therethough and fill a full depth of the aperture.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an overview of a catheter system, including a catheter and hub;

FIG. 2 illustrates a perspective view of the distal region of a catheter having an interlock assembly;

FIG. 3 illustrates an exploded view of the microfabricated tube and marker band of the interlock assembly of FIG. 2;

FIG. 4 illustrates a detailed view of the distal ring of the microfabricated tube of FIG. 3;

FIG. 5 illustrates a detailed view of the marker band of FIG. 3;

FIG. 6 illustrates another embodiment of the marker band in FIG. 5;

FIG. 7 illustrates another embodiment of an interlock assembly;

FIG. 8 illustrates a detailed view of the microfabricated tube of FIG. 7; and

FIG. 9 illustrates a detailed view of the marker band of FIG. 7.

### DETAILED DESCRIPTION

### Overview of Exemplary Catheter Device

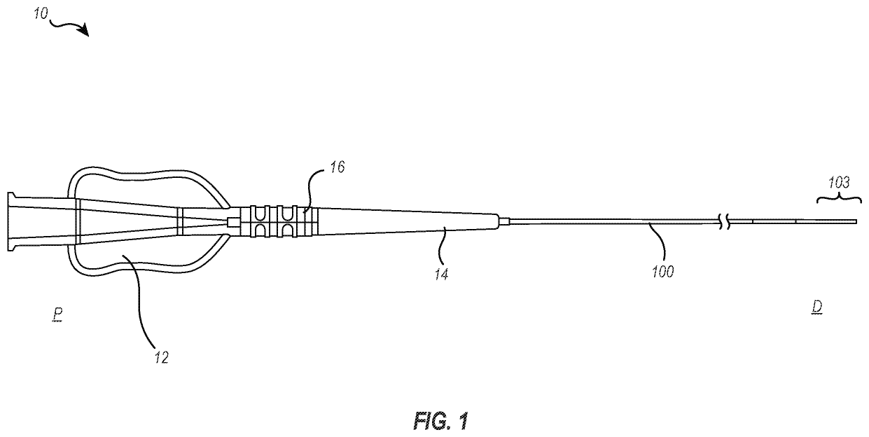

FIG. 1 is an overview of an exemplary catheter device 10 that includes features, described in more detail below, that provide one or more of increased radial strength of a catheter 100, improved connection of components at the distal region 103 of the catheter 100, or enhanced navigation of the catheter 100 to fit into small blood vessels.

The catheter device 10 includes a catheter 100 connected to a hub 12 at a proximal end and extending therefrom to a distal region 103. The catheter 100 may be coupled to the hub 12 using adhesive, a friction fit, through insertion molding, and/or other appropriate attachment means. A strain-relief member 14 can be disposed over the proximal section of the catheter 100 near the hub 12. The illustrated strain-relief member 14 has an outer diameter that substantially matches the adjacent section of the hub 12. The illustrated strain-relief member 14 extends for a distance from the hub 12 with a substantially constant outer diameter before tapering distally to the end where the catheter 100 emerges and extends farther distally. The illustrated strain-relief member 14 may include a groove pattern 16, disposed at the section of substantially constant outer diameter, that functions to provide additional flexibility to the strain-relief member 14 and/or to provide surface features for enhancing user grip and tactile engagement.

The working length of the catheter 100 (i.e., the distance between the distal end of the strain-relief member 14 and the distal region 103 of the catheter 100) may vary according to particular application needs. As an example, the catheter 100 may have a working length of about 50 cm to about 200 cm, though shorter or longer lengths may be utilized where appropriate. The catheter size (typically referring to the inner diameter/lumen size) may also vary according to particular application needs. Examples include 0.010 inches, 0.013 inches, 0.017 inches, 0.021 inches, 0.027 inches, 0.030 inches, 0.035 inches, 0.038 inches, 0.045 inches, 0.065 inches, 0.085 inches, 0.100 inches, or a range including any two of the foregoing values as endpoints. The inside diameter of the catheter can taper from a smaller distal portion to a larger proximal portion. Smaller or larger sizes may be utilized in some applications as appropriate.

Although the distal section of the catheter 100 is shown in this example as having a straight shape, other embodiments may include a shaped distal tip. For example, the distal section of the catheter 100 may have an angled shape, a curved shape (e.g., 45 degree angle, 90 degree angle, J shape, etc.), a compound curved shape, or other appropriate angled or bent shape as known in the art.

The catheter device 10 described herein may be utilized for a variety of interventional applications, including in cardiovascular, peripheral vascular, and neurovascular interventional procedures. Examples include accessing distal anatomy, crossing vessel lesions or blood clots, ischemic treatments, delivering therapeutic agents (e.g., embolic coils or other embolic agents), injecting diagnostic agents (e.g., contrast media or saline), retrieval applications, aspiration applications, or other applications where microcatheter use is beneficial. Distal features of the catheter 100 are described in greater detail below.

### Overview of Interlock Assembly

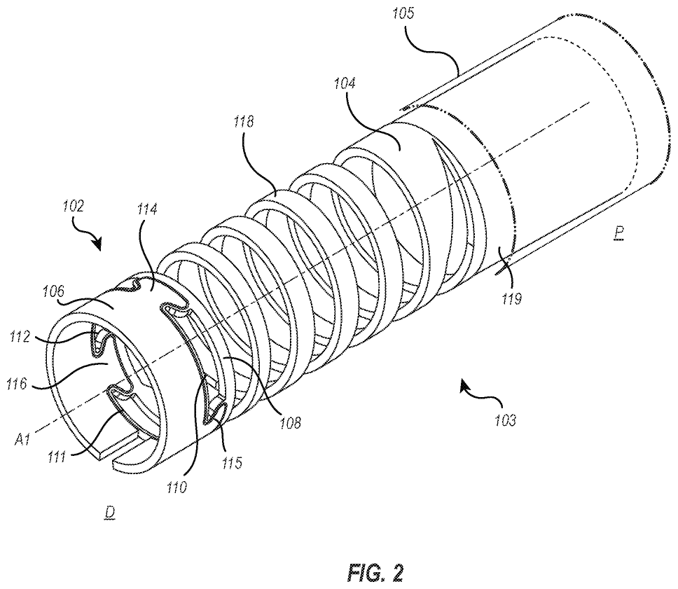

FIG. 2 illustrates the distal region 103 of a catheter 100 including an interlock assembly 102. The outer surface of the catheter 100 may be coated with an appropriate coating material, such as a hydrophilic coating, to form an outer laminate 105 and make the surface more lubricious. The coating material or outer laminate 105 may cover substantially all of the working length of the catheter 100, or a portion thereof. For example, the coating material may be applied to the distal-most 30% to 80% of the working length of the catheter 100. The illustration of FIG. 2 omits outer laminate 105 at the distal end sections to better illustrate internal components, though it will be understood that the laminate 105 can extend across all or a portion of the illustrated portions.

The interlock assembly 102 of the catheter 100 comprises a microfabricated tube 104 and a marker band 106 that improves the connection of components at the distal region 103 of the catheter 100. The microfabricated tube 104 comprises a main segment 119 and a fenestrated segment (shown in this embodiment as a helical segment 118, but which can additionally or alternatively include other fenestrated structures) that longitudinally follow a cylindrical or longitudinal axis A1 of the catheter 100. The helical segment 118 extends from the main segment 119 to a distal ring 108 of the microfabricated tube 104. In some embodiments, the tube 104 can include a superelastic alloy such as nitinol, one or more other metals, alloys, or polymers. The microfabricated tube 104 can be formed from nitinol. In an embodiment, the main segment 119 is formed from stainless steel or polymer material (e.g., polyether ether ketone (PEEK)) and the helical segment 118 (or other fenestrated segment) is formed from nitinol. These materials have been found to provide effective axial response, effective distribution of bending forces, and a smooth bending stiffness profile.

In the illustrated embodiment, the distal ring 108 comprises ring tenons 110, 111, 112 that are configured to engage with band tenons 114, 115, 116 of the marker band 106 to assist in securing the marker band 106 to the distal ring 108. One skilled in the art will recognize that the distal ring 108 may comprise one or more (e.g., two or more) ring tenons 110, 111, 112 and that the marker band 106 may comprise one or more (e.g., two or more) band tenons 114, 115, 116 to enable a secure connection between the marker band 106 and the distal ring 108. One skilled in the art will recognize that the shape of the ring tenons 110, 111, 112 and band tenons 114, 115, 116 may resemble a flange, as depicted in FIG. 2, or another shape that interlocks and restrict movement in a direction parallel to the longitudinal axis A1.

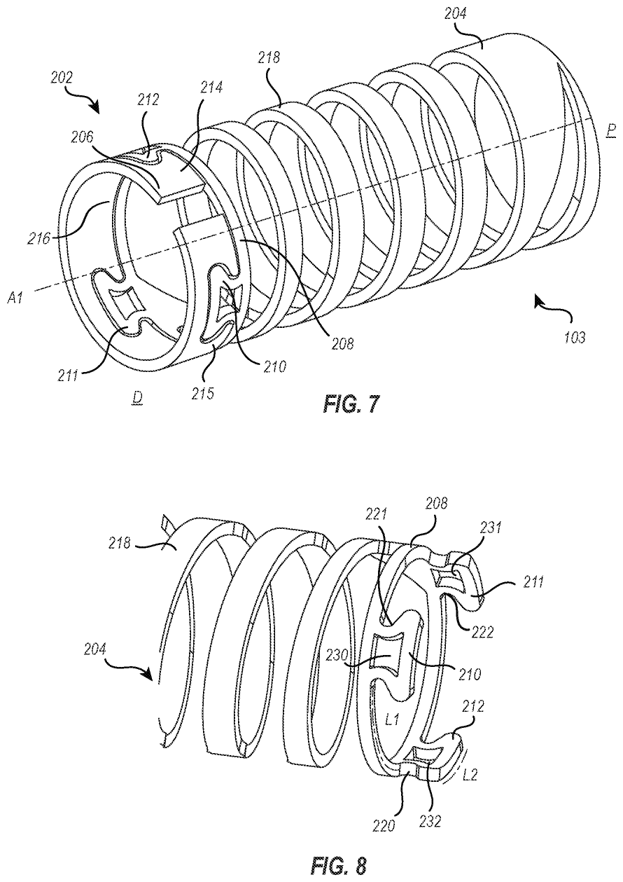

In the illustrated embodiment, the ring tenons 110, 111, 112 are “long” tenons, and the band tenons 114, 115, 116 are “short” tenons as those terms are defined below. In other embodiments, the ring tenons may be short tenons while the band tenons are long tenons (sec, e.g., the embodiment illustrated in FIGS. 7-9). In other embodiments, the ring tenons are substantially equal in arcuate/circumferential length to the band tenons.

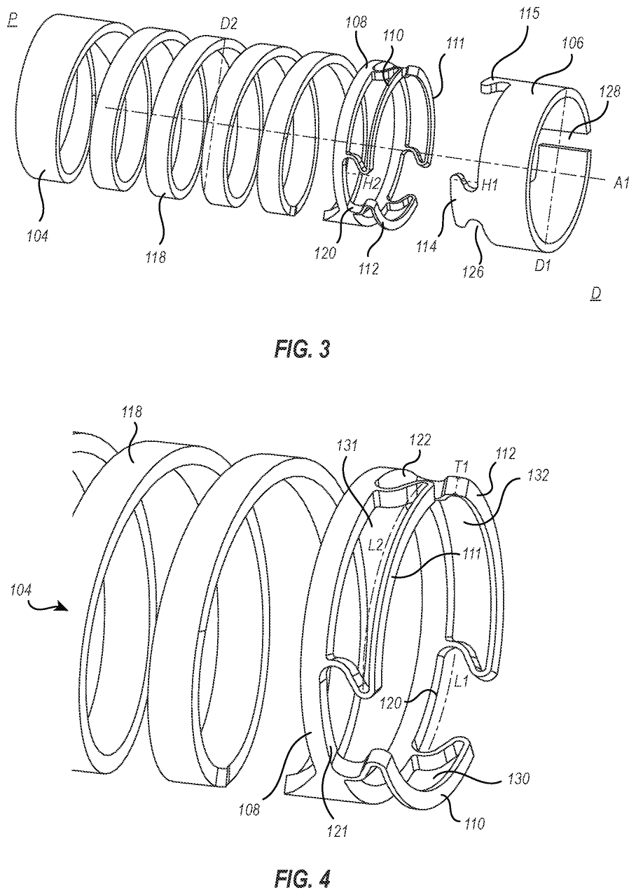

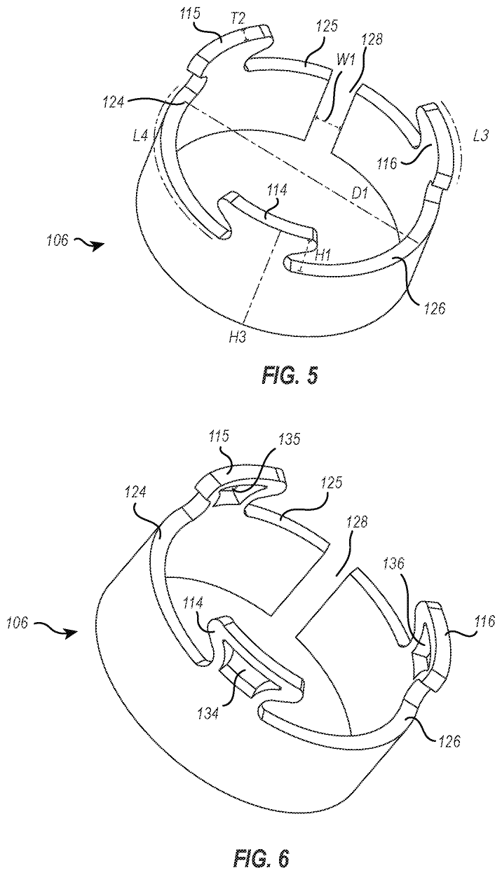

FIG. 3 illustrates the microfabricated tube 104 and the marker band 106 of the interlock assembly 102 in an exploded view, FIG. 4 further illustrates the structure of the distal ring 108, and FIG. 5 further illustrates the marker band 106. In the illustrated embodiment, the distal ring 108 comprises ring mortises 120, 121, 122 to receive the band tenons 114, 115, 116. The marker band 106 comprises band mortises 124, 125, 126 to receive the ring tenons 110, 111, 112. As observed in FIG. 3, the tenon height H1 is equal to the corresponding mortise height H2 to fit tenons 110, 111, 112, 114, 115, 116 to corresponding mortises 120, 121, 122, 124, 125, 126.

In the illustrated embodiment, the marker band 106 has a first diameter D1 that may be crimped down to a second diameter D2, wherein the second diameter D2 corresponds to the diameter of the microfabricated tube 104 and distal ring 108. The marker band 106 comprises a gap 128 to facilitate the crimping of the expanded first diameter D1 to the second diameter D2 of the microfabricated tube 104 and distal ring 108. In an embodiment, the gap 128 is formed between two band tenons 115, 116 (e.g., is coincident with a mortise and not coincident with any band tenons).

The distal ring 108 is formed as a monolithic structure with the microfabricated tube 104 at the distal end of the helical segment 118 and outlines a continuous structure about the longitudinal axis A1. Relative to a fenestrated segment (such as a standard coil) that does not include a distal ring, the distal ring 108 increases hoop strength of the distal tip of the catheter 100 and reduces the risk of ovalization or flattening. The distal ring 108 comprises two or more ring tenons 110, 111, 112 that partially curve about the longitudinal axis A1. The ring mortises 120, 121, 122 are formed between the ring tenons 110, 111, 112 and also partially curve about the longitudinal axis A1. The arcuate length L1 of the ring mortises 120, 121, 122 is equal to the arcuate length L3 of the corresponding band tenons 114, 115, 116, respectively, as shown in FIG. 5. The arcuate length L2 of the ring tenons 110, 111, 112 is likewise equal to the arcuate length L4 of the corresponding band mortises 124, 125, 126. In an embodiment, the arcuate length L1 of the ring mortises 120, 121, 122 is less than the arcuate length of the ring tenons 110, 111, 112, with the arcuate lengths of the band mortises and band tenons corresponding thereto. In another embodiment, the arcuate length of the ring mortises is greater than the arcuate length of the ring tenons, with the arcuate lengths of the band mortises and band tenons corresponding thereto.

The ring tenons 110, 111, 112 comprise radial apertures 130, 131, 132. The radial apertures 130, 131, 132 reduce the surface area about the outer or second diameter D2 of the distal region 103 of the catheter 100. The apertures 130, 131, 132 also allow the material of the outer laminate 105 to flow into the apertures 130, 131, 132 while still enabling the ring tenons 110, 111, 112 to have sufficient structure and shape to interlock with the marker band 106. Allowing the material of the outer laminate 105 to flow into and through the apertures beneficially enables effective coupling of outer and inner laminate layers (e.g., outer laminate to an inner liner) at the distal end, forming a finished laminate structure which further helps to encase and integrate the marker band 106 with other components of the device.

In an embodiment, the marker band 106 is formed from a “radiopaque” material as that term is understood in the intravascular device arts. For example, a radiopaque material may be formed from a material that is more radiopaque than stainless steel. Examples include tantalum, platinum, iridium, tungsten, other highly radiopaque metals, and alloys thereof. The marker band 106 provides an indication of the location of the distal region 103 of the catheter 100. As disclosed above, the marker band 106 can comprise one or more band tenons 114, 115, 116 that are configured to engage with ring tenons 110, 111, 112 of the distal ring 108 and secure the marker band 106 to the distal ring 108. One skilled in the art will recognize that the band tenons 114, 115, 116 may resemble a flange to interlock with the distal ring 108 and restrict movement in a direction parallel to the longitudinal axis A1.

As observed in FIG. 5, the tenon height H1 is less than the cylindrical height H3 of the marker band 106. The marker band 106 has a first diameter D1 that may be crimped down to a second diameter D2. The illustrated marker band 106 comprises a gap 128 having a first width WI that may be reduced and facilitate the crimping of the expanded first diameter D1 to the second diameter D2 of the microfabricated tube 104 and distal ring 108. The adjustable gap 128 allows the band tenons 114, 115, 116 to interlock with the mortises 120, 121, 122 of the microfabricated tube 104. The one or more band tenons 114, 115, 116 partially curve about the longitudinal axis A1. The band mortises 124, 125, 126 are formed between the band tenons 114, 115, 116 and also partially curve about the longitudinal axis A1. The arcuate length L3 of the band tenons 114, 115, 116 is equal to the arcuate length L1 of the corresponding ring mortises 120, 121, 122, respectively, of the distal ring 108 as shown in FIG. 4. The arcuate length L4 of the band mortises 124, 125, 126 are likewise equal to the arcuate length L2 of the corresponding ring tenons 110, 111, 112 of the distal ring 108. In an embodiment, the arcuate length L3 of the band tenons 114, 115, 116 is less than the arcuate length of the band mortises 124, 125, 126. In another embodiment, the arcuate length of the band tenons is greater than the arcuate length of the band mortises.

FIG. 6 illustrates an alternative configuration of the marker band 106 comprising radial apertures 134, 135, 136. The radial apertures 134, 135, 136 reduce the surface area about the outer or second diameter D1 of the distal region 103 of the catheter 100. The apertures 134, 135, 136 allow the material of the outer laminate 105 to flow into and through the apertures 134, 135, 136 and can thereby assist to secure the marker band 106 in place with the microfabricated tube 104. Movement in the longitudinal and radial directions of the catheter are restricted by the outer laminate 105 material integrating with the marker band 106 through the apertures 134, 135, 136, thereby assisting to create a secure connection to prevent dislodgement of the marker band 106. The apertures can additionally or alternatively increase the surface area for coupling between the outer laminate 105 and an inner liner. The marker band (as shown in FIG. 6), the distal ring (as shown in FIG. 4), or both can include such apertures.

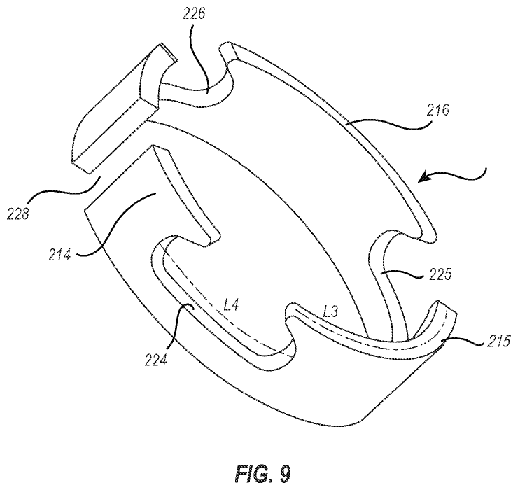

FIG. 7 illustrates an embodiment of interlock assembly 202 at the distal region 103 of the catheter 100. FIG. 8 further illustrates the distal ring 208, and FIG. 9 further illustrates the structure of the marker band 206 of the interlock assembly 202. It is to be understood that interlock assembly 202 is an alternative embodiment of interlock assembly 102 and all the above description is therefore applicable to interlock assembly 202, and vice versa, except for the differences specified. The interlock assembly 202 comprises a microfabricated tube 204 and a marker band 206. The microfabricated tube 204 comprises a helical segment 218 (and/or other fenestrated segment) that longitudinally follows a cylindrical or longitudinal axis A1 of the catheter 100. The helical segment 218 extends from the main segment of the microfabricated tube 204 to a distal ring 208. The helical segment 218 may comprise a left-handed screw or right-handed screw structure and include a uniform or variable pitch depending on the intended application to provide effective axial response, good distribution of bending forces, and a smooth bending stiffness profile and minimizes abrupt changes in stiffness. In one embodiment, the microfabricated tube 204 is formed from nitinol and/or other superelastic material.

The distal ring 208 comprises ring tenons 210, 211, 212 that are configured to engage with band tenons 214, 215, 216 of the marker band 206 and secure the marker band 206 to the distal ring 208. One skilled in the art will recognize that the distal ring 208 may comprise one or more (e.g., two or more) ring tenons 210, 211, 212 and that the marker band 206 may comprise one or more (e.g., two or more) band tenons 214, 215, 216 to enable a secure connection between the marker band 206 and the distal ring 208. The shapes of the ring tenons 210, 211, 212 and band tenons 214, 215, 216 are formed to interlock and restrict movement of the marker band 206 in a direction parallel to the longitudinal axis A1.

The distal ring 208 comprises ring mortises 220, 221, 222 to receive the band tenons 214, 215, 216. The marker band 206 comprises band mortises 224, 225, 226 to receive the ring tenons 210, 211, 212. In this embodiment, the distal ring 208 is formed as a monolithic structure with the microfabricated tube 204 at the distal end of the helical segment 218 and outlines a continuous structure about the longitudinal axis A1. As with earlier embodiments, the distal ring 208 can beneficially increase hoop strength of the distal tip of the catheter 100 (relative to a device omitting such a distal ring, such as a device with a conventional distal coil structure) and reduces the risk of ovalization or flattening.

The illustrated distal ring 208 comprises one or more ring tenons 210, 211, 212 that partially curve about the longitudinal axis A1. The ring mortises 220, 221, 222 are formed between the ring tenons 210, 211, 212 and also partially curve about the longitudinal axis A1. The arcuate length L1 of the ring mortises 220, 221, 222 is equal to the arcuate length L3 of the corresponding band tenons 214, 215, 216 of the marker band 106 in FIG. 9. The arcuate length L2 of the ring tenons 210, 211, 212 is likewise equal to the arcuate length L4 of the corresponding band mortises 224, 225, 226 of the marker band 206. In an embodiment, the arcuate length L1 of the ring mortises 220, 221, 222 is greater than the arcuate length of the ring tenons 210, 211, 212. Alternatively, the arcuate length of the ring mortises may be less than the arcuate length of the ring tenons. One skilled in the art will understand that the sizes and shapes of the tenons may vary so long as the mortises are shaped to receive the corresponding tenons and restrict movement in the longitudinal direction along the longitudinal axis A1.

The ring tenons 210, 211, 212 can comprise radial apertures 230, 231, 232. The radial apertures 230, 231, 232 reduce the surface area about the outer diameter of the distal region 103 of the catheter 100. The apertures 230, 231, 232 allow the material of the outer laminate 105 to flow into the apertures 230, 231, 232 while still enabling the ring tenons 210, 211, 212 to have sufficient structure and shape to interlock with the marker band 206. Movement in the longitudinal and radial directions of the catheter may be further restricted by the outer laminate 105 material better integrating with an inner liner (not shown) and/or the marker band 206 by flowing through the apertures 230, 231, 232 and thereby assisting to create a secure connection to prevent dislodgement of the marker band 206.

In an embodiment, the marker band 206 is formed of tantalum. Other embodiments may additionally or alternatively include one or more other radiopaque materials. The marker band 206 provides an indication of the location of the distal region 103 of the catheter 100. The marker band 206 comprises one or more band tenons 214, 215, 216 that are configured to engage with ring tenons 210, 211, 212 of the distal ring 208 and secure the marker band 206 to the distal ring 208. One skilled in the art will recognize that the marker band 206 may comprise one or more ring tenons 214, 215, 216 and that the shape of the ring tenons 214, 215, 216 may resemble a flange to interlock with the distal ring 208 and restrict movement in a direction parallel to the longitudinal axis A1.

The marker band 206 comprises a gap 228 that may be crimped to allow the tenons 214, 215, 216 of the marker band 206 to interlock with the mortises 220, 221, 222 of the distal ring 208. The gap 228 is formed between band mortises 224, 226 and through a tenon 214 in a direction parallel to the longitudinal axis A1. The marker band 206 comprises two or more tenons 214, 215, 216 that partially curve about the longitudinal axis A1. The band mortises 224, 225, 226 are formed between the tenons 214, 215, 216 and also partially curve about the longitudinal axis A1. The arcuate length L3 of the tenons 214, 215, 216 is equal to the arcuate length L1 of the corresponding ring mortises 220, 221, 222 of the distal ring 208 in FIG. 8. The arcuate length L4 of the band mortises 224, 225, 226 are likewise equal to the arcuate length L2 of the corresponding tenons 210, 211, 212 of the distal ring 208. In an embodiment, the arcuate length L3 of the tenons 214, 215, 216 is greater than the arcuate length of the band mortises 224, 225, 226, respectively, of the marker band 206, though this may be reversed in other embodiments.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

As used herein, the term “microfabricated” refers to any fabrication process capable of manipulating a stock material to form a catheter device having one or more of the features disclosed herein, including any fabrication process capable of forming gaps in an inner shaft as disclosed herein. Examples include, but are not limited to, laser cutting and blade cutting.

The term “tenon” refers to a projecting member on a piece of material for insertion into a mortise to make a joint or connection between individual and distinct components. As used herein, the term “tenon” is also intended to refer to a tab, knob, key, projection, pin, or tongue to fit within a corresponding mortise. The tenon is defined as “short” if the arcuate length of the tenon is less than the opposing and interlocking tenons of the joining piece. The tenon is defined as the “long” if the arcuate length of the tenon is greater than the opposing and interlocking tenons of the joining piece.

The term “mortise” refers to a slot into which some other part of an arrangement of parts fits or passes, such as a cavity formed or carved into a piece of material to receive a tenon. As used herein, the term “mortise” is also intended to refer to a socket, lock, slot, recess, or hole in a portion to receive a corresponding tenon.

For any given element of component of a described embodiment, any of the possible alternatives listed for that element or component may generally be used individually or in combination with one another, unless implicitly or explicitly stated otherwise.

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, a cutting pattern for the outer tube, a coil arrangement, or core joint feature that is not specifically described as being included in the disclosed intravascular device may be optionally excluded (i.e., essentially omitted or completely omitted).

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An interlock assembly for a catheter, comprising:
a microfabricated tube comprising a distal ring, the distal ring comprising at least one ring tenon and at least one ring mortise, wherein the microfabricated tube comprises a fenestrated segment extending from a main segment of the microfabricated tube to the distal ring of the microfabricated tube; and
a marker band comprising at least one band tenon and at least one band mortise,
wherein the at least one ring mortise is configured to receive and interlock with the at least one band tenon and wherein the at least one band mortise is configured to receive and interlock with the at least one ring tenon.

2. The interlock assembly of claim 1, wherein the fenestrated segment is a helical segment.

3. The interlock assembly of claim 2, wherein the distal ring is integrally connected to the helical segment to form a monolithic structure.

4. The interlock assembly of claim 1, wherein the marker band comprises at least two band mortises and the distal ring comprises at least two ring tenons.

5. The interlock assembly of claim 1, wherein the marker band comprises at least two band tenons and the distal ring comprises at least two ring mortises.

6. The interlock assembly of claim 1, wherein the at least one ring tenon comprises an aperture to allow material of an outer laminate to extend therethough.

7. The interlock assembly of claim 1, wherein the at least one band tenon comprises a radial aperture to allow material of an outer laminate to extend therethough.

8. The interlock assembly of claim 1, wherein the marker band comprises a radiopaque material.

9. The interlock assembly of any claim 1, wherein the at least one ring tenon and the at least one band tenon partially curve about a longitudinal axis of the microfabricated tube.

10. The interlock assembly of claim 1, wherein an arcuate length of the at least one ring tenon is greater than an arcuate length of the at least one band tenon.

11. The interlock assembly of claim 1, wherein an arcuate length of the at least one ring tenon is less than an arcuate length of the at least one band tenon.

12. The interlock assembly of claim 9, wherein the marker band comprises a gap that extends in a direction substantially parallel to the longitudinal axis, the gap being disposed so as to not be coincident with a band tenon.

13. The interlock assembly of claim 12, wherein the marker band comprises an expanded outer diameter that is contractable to the diameter of the distal ring.

14. An interlock assembly for a catheter, comprising:
a microfabricated tube comprising a distal ring, the distal ring comprising at least two ring tenons and at least two ring mortises, wherein the microfabricated tube comprises a fenestrated segment extending from a main segment of the microfabricated tube to the distal ring of the microfabricated tube, wherein the fenestrated segment is a helical segment, and wherein the distal ring is integrally connected to the helical segment to form a monolithic structure; and
a marker band comprising at least two band tenons and at least two band mortises,
wherein each ring mortise is configured to receive and interlock with a corresponding band tenon and wherein each band mortise is configured to receive and interlock with a corresponding ring tenon,
wherein at least one ring tenon or at least one band tenon comprises a radial aperture, and wherein an outer laminate extends through the radial aperture.

15. The interlock assembly of claim 14, wherein each ring tenon and each band tenon comprise a radial aperture, and wherein the outer laminate extends through each radial aperture.

16. The interlock assembly of claim 14, wherein the marker band comprises a radiopaque material.

17. The interlock assembly of claim 14, wherein the ring tenons and the band tenons partially curve about a longitudinal axis of the microfabricated tube.

18. The interlock assembly of claim 14, wherein arcuate lengths of the ring tenons are greater than arcuate lengths of the band tenons.

19. The interlock assembly of claim 14, wherein arcuate lengths of the ring tenons are less than arcuate lengths of the band tenons.

20. The interlock assembly of claim 14, wherein the marker band comprises an expanded outer diameter that is contractable to the diameter of the distal ring.
